(19) United States
(12) Patent Application Publication
(10) Pub. No.: US 2011/0039815 A1
(43) Pub. Date: Feb. 17, 2011

(54) MACROCYCLIC METAL COMPLEXES FOR THEIR USE AS ANTICANCER AGENTS

(76) Inventors: Wiley J. Youngs, Akron, OH (US); Claire A. Tessier, Akron, OH (US); Doug A. Medvetz, Elyria, OH (US); Michael J. Taschner, Akron, OH (US)

Correspondence Address: Joseph J. Crimaldi;Roetzel & Andress, 222 S. Main St., Akron, OH, 44308, US

(21) Appl. No.: 11/917,660

(22) PCT Filed: Jun. 14, 2006
(86) PCT No.: PCT/US06/23137

§ 371 Date: Nov. 5, 2010

Related U.S. Application Data
(60) Provisional application No. 60/690,536, filed on Jun. 14, 2005.
(60) Provisional application No. 60/785,311, filed on Mar. 23, 2006.

(51) Int. Cl. A61K 31/555; A61P 35/00; C07D 285/00; C07F 15/00

(52) U.S. Cl. 514/184; 540/467; 540/465

(57) ABSTRACT

In one embodiment the present invention relates to a method of treating cancerous cells in a mammal comprising the steps of administering to the cancerous cells an effective amount of a cyclic amine wherein the cyclic amine contains sulfur or nitrogen and the structure includes an interchealted metal ion.

### RELATED APPLICATION DATA

This application claims priority to previously filed U.S. Provisional Application No. 60/690,536, filed on Jun. 14, 2005 entitled “Macrocyclic Metal Complexes for their Use as Anticancer Agents” and to previously filed U.S. Provisional Application No. 60/785,311, filed on Mar. 23, 2006 entitled “Macrocyclic Metal Complexes for their Use as Anticancer Agents”. Both are herby incorporated by reference in their entirety.

### BACKGROUND OF THE INVENTION

Interest has increased in recent years in anticancer chemotherapy using metal chelates. Although a wide range of metal complexes have been studied, platinum compounds have been the most successful anticancer agents. Among the more notable successes is the platinum-based drug Cisplatin.

A central issue in cancer chemotherapy is the severe toxic side effects of the anticancer agents on healthy tissues, which invariably imposes dose limitations, treatment delay or even discontinuance of therapy. Notably, Cisplatin causes cytotoxicity to normal cells at a rate that is nearly equal to the cancerous cells, which leads to severe side effects such as extreme weight loss, vomiting, and/or even death. Thus, there is a need in the art for anticancer chemotherapeutic agents that preferentially attack cancer cells while leaving non-cancerous cells comparatively unharmed.

### SUMMARY OF THE INVENTION

The present invention generally relates to the use of multidentate ligands for treating cancers. More specifically, the present invention relates to using families of macrocyclic ligands having sulfur and/or nitrogen hetero atoms for treating cancers, wherein the sulfur and nitrogen atoms are complexed to rhodium (Ill) and ruthenium (III). The present invention comprises two families of rhodium complexes that display anticancer activity, while leaving non-cancerous cells comparatively unharmed.

It is therefore an aspect of the present invention to provide a method of treating cancerous cells in a mammal comprising the step of administering to the cancerous cells an effective amount of a cyclic amine comprising the structure:

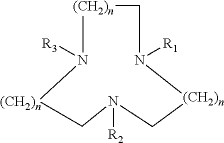

wherein each R is independently selected from the group consisting of a proton, an alkyl, an ether, an alcohol, a carboxylic acid, an aryl, an amino acid, or a peptide, wherein each n varies independently and is an integer equal to either 1 or 2, and wherein the structure further comprises an interchelated metal ion selected from the group consisting of iron, ruthenium, osmium, cobalt, rhodium, iridium, nickel, palladium, platinum, copper, chromium, gallium, molybdenum, manganese, and tungsten.

A preferred composition of the cyclic amine includes a cyclic amine rhodium(III)-trichloride complex wherein R is selected from the group consisting of a proton, an alkyl, an ether, an alcohol, a carboxylic acid, an aryl, an amino acid, and a peptide and wherein n is either 1 or 2.

Another aspect of the present invention is to provide a method of treating cancerous cells in a mammal comprising the step of administering to the cancerous cells an effective amount of a thiaether comprising the structure:

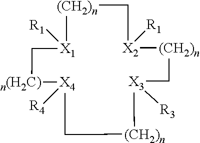

wherein each R is independently selected from the group consisting of a proton, an alkyl, an ether, an alcohol, a carboxylic acid, an aryl, an amino acid, a peptide, or null, wherein each X is independently either sulfur or nitrogen, when any X is sulfur the corresponding R is null, and wherein the structure further comprises an interchelated metal ion selected from the group consisting of iron, ruthenium, osmium, cobalt, rhodium, iridium, nickel, palladium, platinum, copper, chromium, gallium, molybdenum, manganese, and tungsten.

A preferred composition of the thiaether compound includes a rhodium(III)-trichloride complex wherein R is selected from the group consisting of a proton, an alkyl, an ether, an alcohol, a carboxylic acid, an aryl, an amino acid, a peptide, and null and wherein X is either sulfur or nitrogen; and if X is sulfur, R is null.

Another aspect of the present invention is to provide a method of treating cancerous cells in a mammal comprising the step of administering to cancerous cells a thiaether rhodium(III)-trichloride complex comprising the structure:

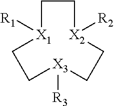

wherein R is selected from the group consisting of a proton, an alkyl, an ether, an alcohol, a carboxylic acid, an aryl, an amino acid, a peptide, and null, wherein X is either sulfur or nitrogen and when X is sulfur R is null.

Another aspect of the present invention is to provide a chemical composition comprising:

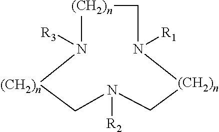

wherein each R is independently selected from the group consisting of a proton, an alkyl, an ether, an alcohol, a carboxylic acid, an aryl, an amino acid, or a peptide, wherein each n varies independently and is an integer equal to either 1 or 2, and wherein the structure further comprises an interchelated metal ion selected from the group consisting of iron, ruthenium, osmium, cobalt, rhodium, iridium, nickel, palladium, platinum, copper, chromium, gallium, molybdenum, manganese, and tungsten.

A preferred composition of the previous structure involves a structure comprising an interchelated Rh(III) metal bound to one or more nitrogen atoms present.

Another embodiment of the structure involves a composition comprising an interchelated Ru(III) metal bound to one or more nitrogen atoms present.

Another aspect of the present invention is to provide a chemical composition comprising:

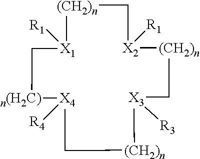

wherein each R is independently selected from the group consisting of a proton, an alkyl, an ether, an alcohol, a carboxylic acid, an aryl, an amino acid, a peptide, or null, wherein each X is independently either sulfur or nitrogen, when any X is sulfur the corresponding R is null, and wherein the structure further comprises an interchelated metal ion selected from the group consisting of iron, ruthenium, osmium, cobalt, rhodium, iridium, nickel, palladium, platinum, copper, chromium, gallium, molybdenum, manganese, and tungsten.

A preferred composition of the previous structure involves a structure further comprising an interchelated Rh(III) metal bound to one or more nitrogen atoms present.

Another aspect of the present invention is to provide a chemical composition comprising:

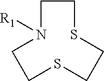

wherein the structure further comprises an interchelated Rh(III) metal bound to the nitrogen atom and one or both of the sulfur atoms.

Another aspect of the present invention to provide a chemical composition comprising:

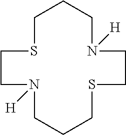

Another aspect of the present invention to provide a composition comprising:

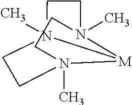

wherein M comprises a metal ion selected from the group consisting of iron, ruthenium, osmium, cobalt, rhodium, iridium, nickel, palladium, platinum, copper, chromium, gallium, molybdenum, manganese, and tungsten.

### BRIEF DESCRIPTION OF THE FIGURES

FIG. 1 is a picture of control wells of the normal ovarian cell line OVepi with no compounds added.

FIG. 2 is a picture of the control wells of the ovarian cancer cell line NUTU-19 with no compounds added.

FIG. 3 is a picture of the effect of a complex synthesized by reacting a cyclic amine ligand with RhCl₃ in ethanol on the ovarian cancer cell line NUTU-19.

FIG. 4 is a picture showing the effect of a complex synthesized by reacting a cyclic amine ligand with RhCl₃ in ethanol on non-cancerous ovarian OVEPI cells.

FIG. 5 is a crystallographical characterization of the compound disclosed in formula 8.

FIG. 6 is a picture of the non-cancerous ovarian cell OVepi wells where one thiaether-RhCl₃ complex was added at 1.54×10⁻³ M.

FIG. 7 is a picture of the ovarian cancer cell line NUTU-19 wells where one thiaether-RhCl₃ complex was added at 1.54×10⁻³M.

FIG. 8 is a picture of the effect of complex synthesized by reacting a thiaether ligand with RhCl₃ in ethanol on the ovarian cancer cell line NUTU-19.

FIG. 9 is a picture showing the effect of a complex synthesized by reacting a thiaether ligand with RhCl₃ in ethanol on non-cancerous ovarian OVEPI cells.

FIG. 10 is a thermal ellipsoid plot for the rhodium-trichloride complex shown in formula 16.

FIG. 11 is a graph of MTT (colorimetric assay) results of two thiaether-RhCl₃ complexes at 1.00×10⁻⁶ M against the non-cancerous ovarian cells known as OVepi.

FIG. 12 is a graph of MIT (colorimetric assay) results of two thiaether-RhCl₃ complexes at 1.00×10⁶M against the ovarian cancer cell line known as NUTU-19.

FIG. 13 crystallographical characterization and a thermal ellipsoid plot (TEP) of formula 19.

### DETAILED DESCRIPTION OF INVENTION

The present invention generally relates to the use of multidentate ligands as anticancer agents. More specifically, the present invention includes without limitation the use of a family of cyclic amine ligands, as well as, the use of a family of thiaether ligands, all of which are bound to rhodium (III), ruthenium (III) or other multivalent metal ions.

The term interchelated metal ion is used as a description of how the metal ion reacts with the cyclic ring. Interchelated being defined as a chemical compound in the form of a heterocyclic ring, containing a metal ion attached by coordinate bonds to at least two nonmetal ions. A broader term that can also be substituted is that of an intercalated metal ion.

### Cyclic Amine Ligands

The cyclic amine ligands used to complex rhodium (III), rhodium (III) trichloride, ruthenium(III) and other metals are generally represented by formula 1:

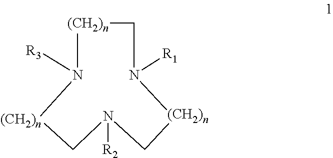

wherein each n is an integer and can vary independently from one to two. Each R group (1 through 3) can vary independently and can be a hydrogen atom, an alkyl such as but not limited to a methyl, an ether such as but not limited to methyl ethyl ether, an alcohol such as but limited to ethanol, methanol or propanol, a carboxylic acid such as but not limited to acetic acid, an aryl such as but not limited to benzene, an amino acid such as but not limited to serine or threonine, or a peptide such as but not limited to luetinizing hormone. The foregoing R groups can be modified or derivatized for increased lipophilicity, increased hydrophilicity, and/or in vivo targeting enhancement for tumor specificity.

In addition to rhodium, other metal-based compounds may be used in the form of alkoxides, bromides, chlorides, or iodides. For example, metals yielding ions within the scope of the present invention include without limitation iron, ruthenium, osmium, cobalt, rhodium, iridium, nickel, palladium, platinum, copper, chromium, gallium, molybdenum, manganese, and tungsten. In general, the characteristics of a suitable metal ion include without limitation stability under physiological conditions. Additionally, suitable metal ions are further characterized by sufficient lipophilicity to be delivered to a locus within the body for which dosing is indicated.

Examples of ligand formula within the scope of the present invention include without limitation formula 2 through 5:

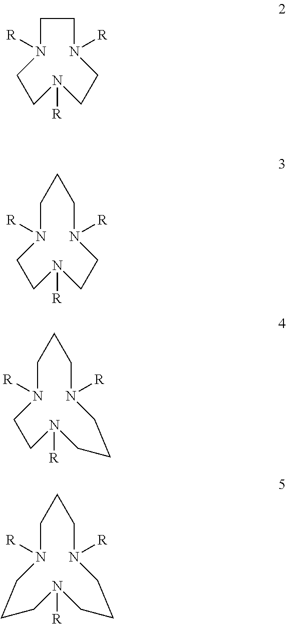

In one embodiment a cyclic amine ligand of formula 2, namely ^{Me3}TacnRhCl₃, tests positive for anticancer activity against the cervical cancer cell line Hela S3. Various concentrations of ^{Me3}TacnRhCl₃ where shown to have anticancer properties, including 1.5×10⁻², 1.5×10⁻³, 1.5×10⁻⁴, and 1.5×10⁻⁶ M. The 10⁻² molar preparation's Hela cell death rate was approximately 93.1% and the death rate of the 10⁻³ molar preparation was approximately 90%.

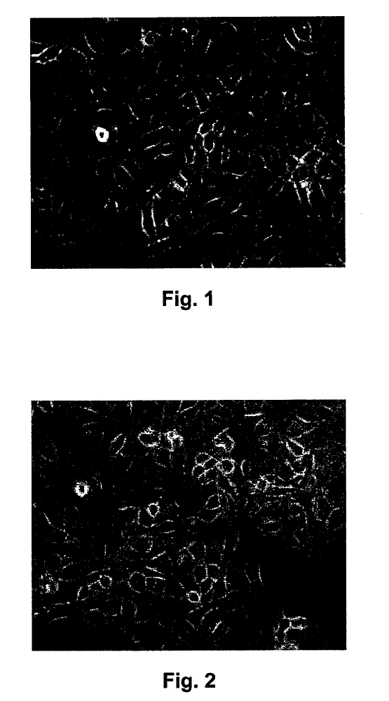

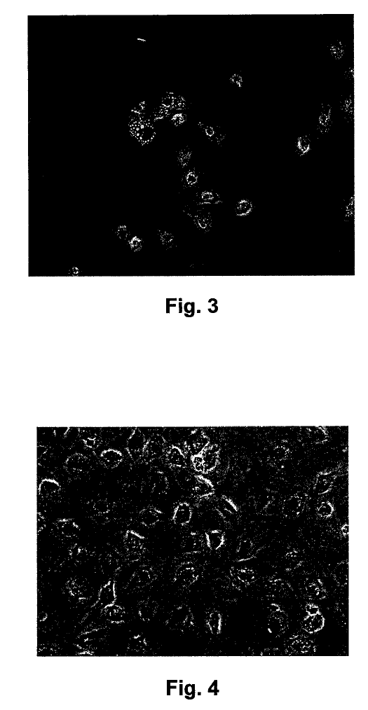

In another embodiment cyclic amines of the present invention are effective in killing the ovarian cancer cell line NUTU-19 while leaving non-cancerous ovarian cells known as OVEPI cells comparatively unharmed. FIGS. 1 and 2 detail controls lacking a cyclic amine-rhodium complex. In FIG. 1 the cells have become attached and viable and have grown to confluency after 48 hour incubation in cell culture media. Notably, the cells remain in a close contact colony. Similarly, in FIG. 2 the cells have become attached and viable and have grown to confluency in 24 hours of incubation in cell culture media. Like the NUTU control cells in FIG. 2, these cells are also in a very close contact colony. In comparison, FIGS. 3 and 4 represent the killing efficacy of the cyclic amine-rhodium complexes against the control NUTU-19 ovarian cancer and non-cancerous OVEPI ovarian cell lines. Specifically, in FIG. 3 the cells were allowed to grow to confluency and then 2 mL of a 1.75×10⁻³ M solution of formula 8 was added and incubated for 24 hours.

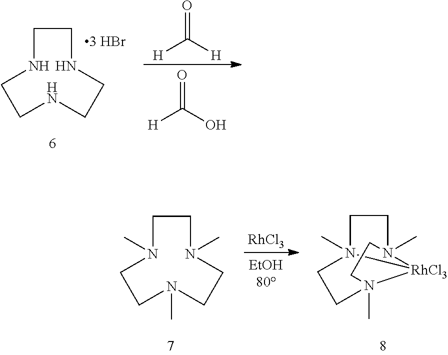

The circular bodies indicate detached, non-viable, or lysed cells. The cells in the middle of FIG. 3 show deleterious morphology changes and internal vesicle formation and elongation in addition to a loss of cell colony contact, which leads to cell lysis. Significantly, in FIG. 4 the non-cancerous OVEPI cells were grown to confluency and then 2 mL of a 1.75×10⁻³ M solution of formula 8 was added and incubated for 24 hours. Notably, the cells continue to show close cell contact, good cell attachment and viability, and a lack of deleterious cell morphology changes. Thus, the cyclic amine-rhodium complex effectively kills cancerous cells while leaving non-cancerous cells comparatively unharmed.

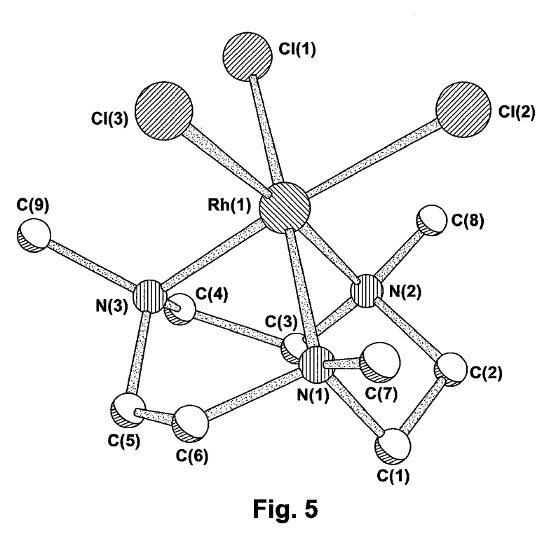

Toxicity studies in rat models of the compound detailed in FIG. 5 were carried out through intravenous injection to determine the LD₅₀ of the ^{Me3}TacnRhCl₃ complex by standard Institutional Animal Care and Use Committee (IUACUC) protocol. The LD₅₀ of this complex was not determined because of the limited solubility in the 0.7% saline solution used. However, approximately 172 mg/kg of complex was injected during a six hour period and showed no complex-related toxicity or side effects.

### Thiaether Ligands

The thiaether ligands of the present invention are represented by the following formula 9:

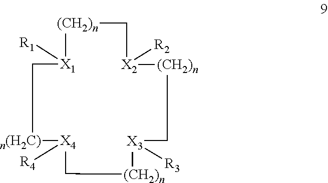

wherein each n is an integer and can vary independently from 1 to 2. Each R group (1-4) can vary independently and can be a hydrogen atom, an alkyl such as but not limited to a methyl, an ether such as but not limited to methyl ethyl ether, an alcohol such as but not limited to ethanol, methanol or propanol, a carboxylic acid such as but not limited to acetic acid, an aryl such as but not limited to benzene, an amino acid such as but not limited to serine or threonine, a peptide such as but not limited to luetinizing hormone, or nothing at all, i.e. null. Each X group (1-4) varies independently and can be either sulfur or nitrogen. Note that when X is sulfur there is no R group attached to X, i.e. the R group becomes null.

In addition to rhodium, other metal-based compounds may be used in the form of alkoxides, bromides, chlorides, or iodides. For example, metals yielding ions within the scope of the present invention include without limitation iron, ruthenium, osmium, cobalt, rhodium, iridium, nickel, palladium, platinum, copper, chromium, gallium, molybdenum, manganese, and tungsten. In general, the characteristics of a suitable metal ion include without limitation stability under physiological conditions. Additionally, suitable metal ions are further characterized by sufficient lipophilicity to be delivered to locus within the body for which dosing is indicated.

Examples of ligand formula within the scope of the present invention include without limitation:

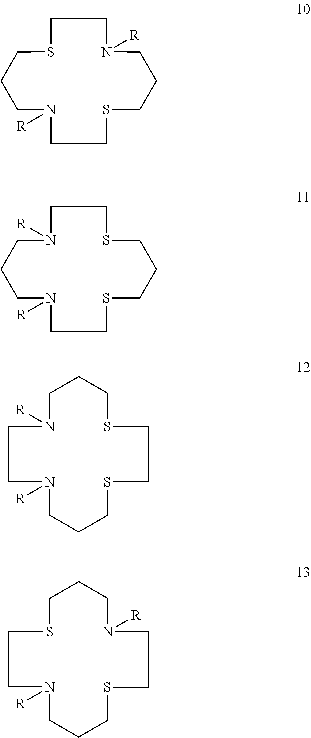

Thiaether-RhCl₃ complexes according to formula 13 were tested against the Hela S3 cell line and showed anticancer activity. The resulting death rate of the 1.5×10⁻³ molar preparation was approximately 93%, showing a killing efficacy similar to that of ^{Me3}TacnRhCl₃.

The foregoing thiaether rhodium complexes have also been tested against the ovarian cancer cell line NUTU-19 for anticancer activity. Preliminary results showed that thiaether Rh(III) complexes had killing efficacy's of approximately 83%. The photos shown in FIGS. 1 through 4 and 6 through 9 were taken after a 24 hour incubation.

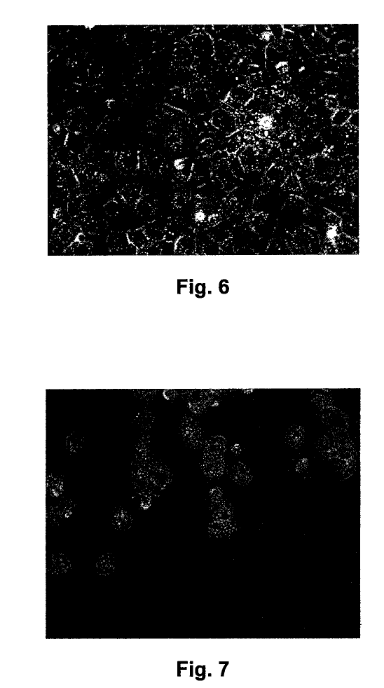

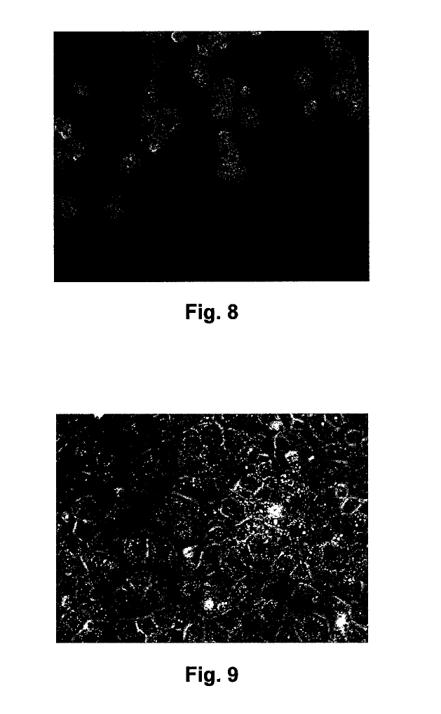

FIGS. 1 and 2 are controls having no thiaether rhodium complex. FIGS. 6, 7, 8, and 9 represent the killing efficacy of the thiaether rhodium complexes against the OVEPI normal ovarian cell lines and NUTU-19 ovarian cancer. Specifically, in FIG. 8 the cells were allowed to grow to confluency and then 2 mL of a 1.54×10⁻³M solution of formula 7 was added. Notably, the cells have become significantly swelled, and major internal vesicle formation and cell morphology changes can be seen. The circular bodies are non-viable, detached, or lysed cells. Similar to the result of the cyclic amine complex shown in FIG. 3 these cells have lost colony contact. In comparison, FIG. 9 shows OVEPI cells grown to confluency where 2 mL of a 1.54×10⁻³ M solution of formula 7 was added and incubated for 24 hours. Here the cells have maintained close cell contact, good morphology and cell attachment, and are viable. Minor vesicle formation is observed, but has not led to cell death.

The thiaether ligands of the present invention are also represented by formula 14:

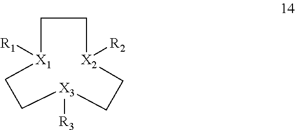

Wherein each R group (1-3) can vary independently and can be a hydrogen atom, an alkyl such as but not limited to a methyl, an ether such as but not limited to methyl ethyl ether, an alcohol such as but not limited to ethanol, methanol or propanol, a carboxylic acid such as but not limited to acetic acid, an aryl such as but not limited to benzene, an amino acid such as but not limited to serine or threonine, a peptide such as but not limited to luetinizing hormone, or null. Each X group (1-3) varies independently and can be either a sulfur or nitrogen. Note when X is sulfur there is no R group attached to X, i.e. the R group becomes null. In addition to rhodium, other metal-based compounds may be used in the form of alkoxides, bromides, chlorides, or iodides. Examples of ligand formula within the scope of the present invention include without limitation:

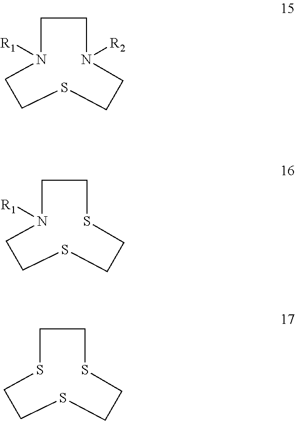

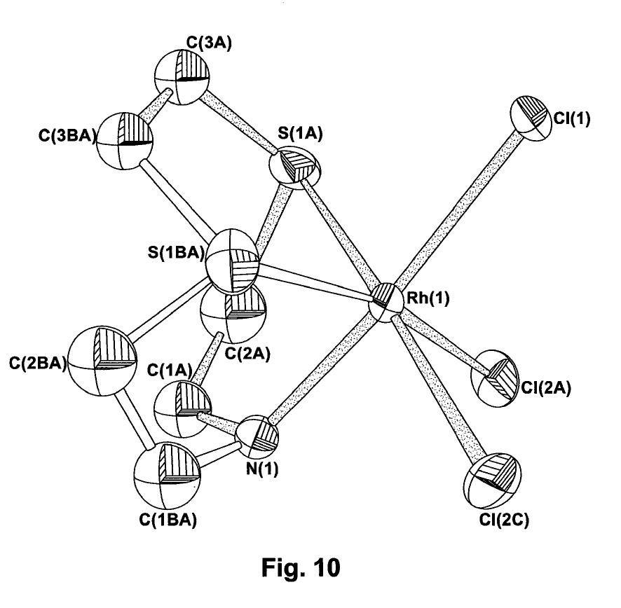

Thiaether-RhCl₃ complexes according to formula 15 and 16 have been tested against the normal ovarian cell line OVepi and the ovarian cancer cell line NUTU-19 and have shown anticancer activity. Formula 16 is further represented in FIG. 10 in a thermal ellipsoid plot for the rhodium-trichloride complex.

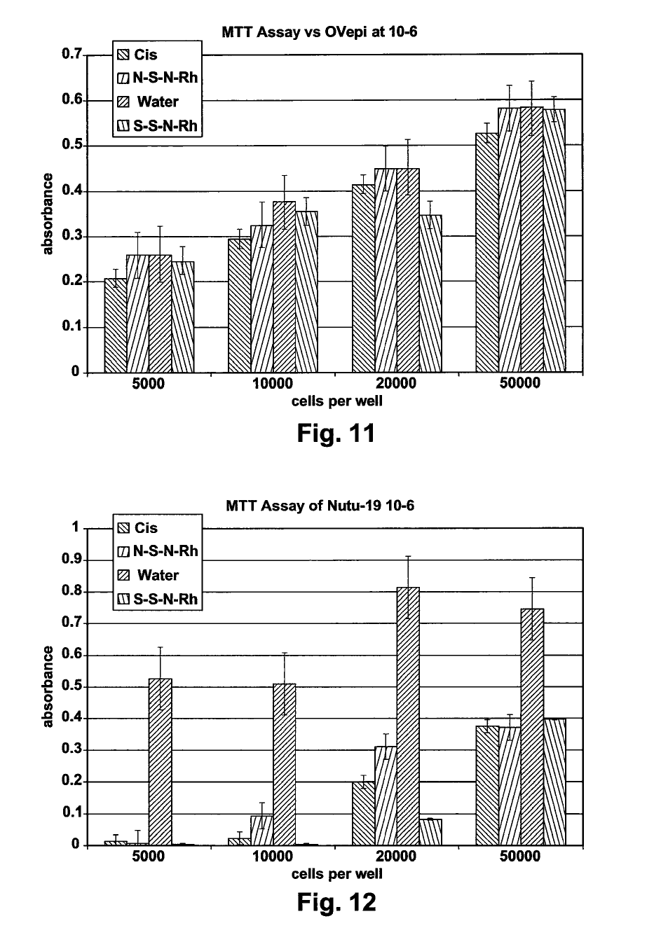

The graphs shown in FIGS. 11 and 12 represent the MTT assay results, where absorbance value is directly related to viable cell concentration, of 15 and 16 rhodium complexes against the OVepi and NUTU-19 cell lines in comparison to Cisplatin. Water is used as a control. Cells were plated at 5,000, 10,000, 20,000, and 50,000 cells per well in triplicate 96-well plates and incubated overnight. Thiaether-RhCl₃ complexes of 15 and 16 were added at 1×10⁻⁶M and incubated overnight. MTT assay protocol, as is known in the art, was then followed.

### Example Synthesis

The synthesis of the rhodium (III) complexes of the present invention can be readily accomplished in relatively high yield in a one-pot synthesis by refluxing the respective ligands with rhodium (III) trichloride in ethanol for 2 hours in accordance with Weiss, B. et al., J. Chem. Ber. 1979, 112, 2220; and Flood, T. et al., Organometallics. 1996, 15, 491-498. The previously synthesized complexes used in this invention are also water soluble and have been found stable in aqueous solutions including physiological saline. This high stability of the rhodium (III) complexes under physiological conditions is crucial to their use in vivo as anticancer agents.

In one embodiment, the cyclic amine ligand from formula 7 (as previously described) was synthesized as outlined using established procedures previously detailed. The reaction of 7 with RhCl₃ in ethanol at reflux for two hours yields 8 at 80% yield. Complex 8, which was crystallographically characterized in FIG. 5, is stable in water and soluble up to 2.1×10⁻² molar in physiological saline solution. The results of the cytotoxicity studies of 16 are reported in the preliminary cell culture results section.

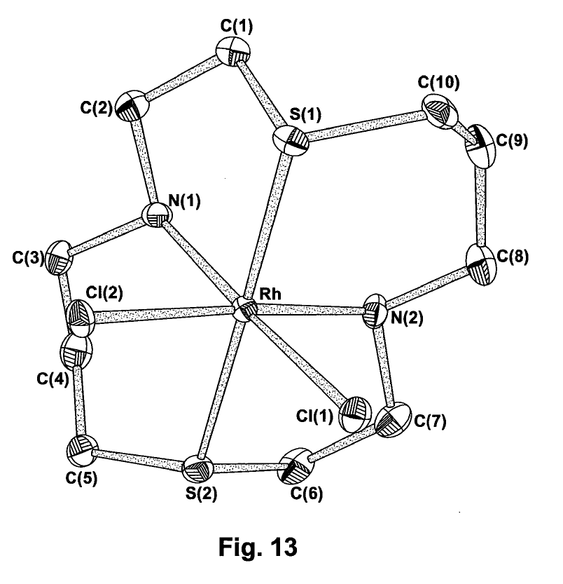

The first thiaether ligand 18 synthesized is a derivative of 13 where both R groups are protons. The synthesis of 18 is outlined below. Compound 18 was then reacted with RhCl₃ in ethanol to give the rhodium complex 19. Compound 19 has been crystallographically characterized as well, and a thermal ellipsoid plot (TEP) structure is shown in FIG. 13.

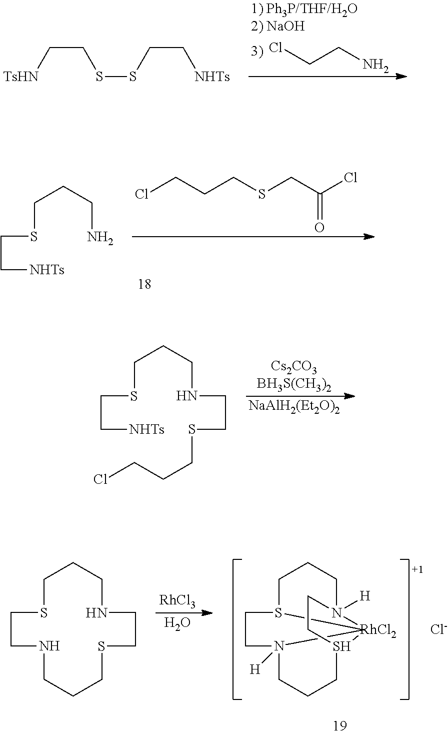

The metal complex in Formula 19 and/or FIG. 13 have been tested against the rat cancer cell line NUTU-19 and the normal rat ovarian cell line OVEPI in vitro in six well plates. As shown in Table 1 the complex of FIG. 13 is very effective at killing cancerous NUTU-19 cells with cell death rates of 87% and 86%, respectively, but kills the normal ovarian cells OVEPI at a much lower rate, with cell death rates of 32% and 29%, respectively. The NUTU-19 cell line is the cancerous version of the OVEPI cell line and is the cell line that will be inoculated into the Fischer 344 rat for the in vivo cancer study. The experiments were run by plating a known number of cells and allowing them to attach overnight. The tests were done in triplicate and in each six well plate, two wells were control wells where cells grew normally, and two wells had aqueous of FIG. 13 added.

TABLE 1

| Cell Type | NUTU-19 | OVEPI |
|---|---|---|
| Death Rate of cells incubated with | 86% | 29% |
| compound of FIG. 13 | | |
| # of cells in control well | 9.69 × 10⁴ | 26.6 × 10⁴ |
| | cells/well | cells/well |
| # of cells left after incubation with | 1.20 × 10⁴ | 18.75 × 10⁴ |
| compound of FIG. 13 | cells/well | cells/well |

Death rates are reported in percentage of total cells killed after 24 hour incubation with FIG. 13 at 1.54×10⁻³ M. The term “# of cells” as used in the table means average cells per well per six well plate after cells were grown to confluency. Similarly, the term “# of cells left after incubation” means the average cells left per well per six well plate after 24 hour incubation of confluent cells with FIG. 13 at 1.54×10⁻³ M. All test wells were incubated with 2 mL of aqueous compound and counted using a hemocytometer.

As shown in FIGS. 1 through 4 and 6 through 9, complexes of FIG. 13 result in substantial cell death and morphological changes to the cancerous NUTU-19 cells while the normal OVEPI cells continue to show strong cell viability and a lack of morphological changes. FIG. 1 represents the control well of the normal ovarian OVepi cells where the cells were allowed to grow normally in culture media. FIG. 6 is a representation of the complex of FIG. 5 tested on the OVepi cell culture at a concentration of 1.54×10⁻³M. FIG. 2 represents the NUTU-19 cell control well where the cells grew normally in the culture media. FIG. 7 is a representation of the complex of FIG. 5 against the NUTU-19 cell culture at the same concentration as FIG. 6. FIG. 3 is a representation of the complex of FIG. 5 tested on the cancerous NUTU-19 cell culture at a concentration of 1.54×10⁻³ M. FIG. 8 represents the test of complexes shown in FIG. 13 on the cancerous NUTU-19 cells at a concentration of 1.75×10⁻³ M. FIGS. 4, 6 and 9 represent the test on the OVEPI cell culture. FIG. 1 represents the normal OVEPI cell control well where the cells grew normally in the culture media. FIG. 4 is a representation of complex of FIG. 5 against the normal OVEPI cell culture at the same concentration as FIG. 3. Lastly, FIG. 9 represents complexes as shown by FIG. 13 tested against the normal OVEPI cell culture at the same concentration as FIG. 8.

### Dosages and Application Methods

The term effective amount defines the dosage needed to effectuate proper treatment. This dosage will vary based on the chemical and physiological make-up of the person/animal treated, the nature and exact location of the cancerous cells and the exact type of cancerous cells being treated. A preferred dosage range for the effective amount is 1-1000 mg/kg, also preferred is the range 10-100 mg/kg, and also preferred is the range 35-65 mg/kg.

The method of application can be but is not limited to intravenous injection, intraperitoneal (i.e. abdominal cavity) injection or oral ingestion. Using the injection method, the drug is dissolved into a suitable solution. One such solution is a physiological sodium chloride solution. Such a solution can be but is not limited to 0.5% to 1.0% sodium chloride in water, a concentration that is of biological significance as it is isotonic with blood plasma. Also significant is the fact that the metal complexes are water and sodium chloride solution soluble. Another suitable solution into which the drug dissolves includes dimethyl sufloxide (DMSO). In addition, other solvents which will solubilize the drug and are compatible with the human/mammal body are acceptable. The oral ingestion method includes a pill, capsule, caplet or tablet. Such an ingestion could be the pure form of the drug or of a lower concentration that has been mixed with a carrier and/or binder known in the art.

The foregoing examples are considered only illustrative of the principles of the invention rather than an exclusive list of embodiments. Further, since numerous modifications and changes will readily occur to those skilled in the art, the invention is not intended to be limited to the exact construction and operation shown and described, and accordingly, all suitable modifications and equivalents are within the scope of the present invention.

What is claimed is:

1. A method of treating cancerous cells in a mammal comprising the step of:
administering to the cancerous cells an effective amount of a cyclic amine comprising the structure: 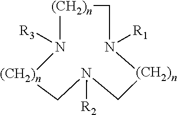
wherein R₁, R₂ and R₃ are independently selected from the group consisting of a proton, an alkyl, an ether, an alcohol, a carboxylic acid, an aryl, an amino acid, or a peptide;
wherein each n varies independently and is an integer equal to either 1 or 2; and
wherein the structure includes an interchelated metal ion selected from the group consisting of iron, ruthenium, osmium, cobalt, rhodium, iridium, nickel, palladium, platinum, copper, chromium, gallium, molybdenum, manganese, and tungsten.

2. The method of claim 1 wherein the cyclic amine comprises ^{Me3}TacnRhCl₃and the metal ion comprises rhodium (III).

3. The method of claim 1 wherein the cyclic amine is a rhodium(III)-trichloride complex and the metal ion is rhodium(III).

4. A method of treating cancerous cells in a mammal comprising the step of:
administering to the cancerous cells an effective amount of a thiaether comprising the structure: 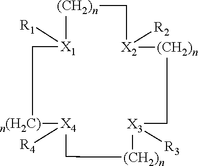
wherein each R₁, R₂, R₃ and R₄ are independently selected from the group consisting of a proton, an alkyl, an ether, an alcohol, a carboxylic acid, an aryl, an amino acid, a peptide, or null;
wherein X₁, X₂, X₃ and X₄ are either sulfur or nitrogen;
wherein if X₁ is sulfur, R₁ is null;
wherein if X₂ is sulfur, R₂ is null;
wherein if X₃ is sulfur, R₃ is null;
wherein if X₄ is sulfur, R₄ is null; and
wherein the structure includes an interchelated metal ion selected from the group consisting of iron, ruthenium, osmium, cobalt, rhodium, iridium, nickel, palladium, platinum, copper, chromium, gallium, molybdenum, manganese, and tungsten.

5. The method of claim 4 wherein X₁ and X₄ are sulfur;
X₂, and X₃ are nitrogen; and
R₁ and R₄ are null.

6. The method of claim 4 wherein the thiether structure is a rhodium(III)-trichloride complex and the metal ion is rhodium(III).

7. The method of claim 6 wherein X₁ and X₄ are sulfur;
X₂, and X₃ are nitrogen; and
R₁ and R₄ are null.

8. The method of claim 6 wherein X₁ and X₂ are sulfur;
X₃ is nitrogen; and
R₁ and R₂ are null.

9. A method of treating cancerous cells in a mammal comprising the step of:
administering to the cancerous cells an effective amount of thiaether rhodium(III)-trichloride complex comprising the structure: 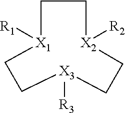
wherein R₁, R₂ and R₃ are selected from the group consisting of a proton, an alkyl, an ether, an alcohol, a carboxylic acid, an aryl, an amino acid, a peptide, and null;
wherein X₁, X₂ and X₃ are either sulfur or nitrogen;
wherein if X₁ is sulfur, R₁ is null;
wherein if X₂ is sulfur, R₂ is null; and
wherein if X₃ is sulfur, R₃ is null.

10. The method of claim 9 wherein X₁ is sulfur;
X₂, and X₃ are nitrogen; and
R₁ is null.

11. The method of claim 9 wherein X₁ and X₂ are sulfur;
X₃ is nitrogen; and
R₁ and R₂ are null.

12. A chemical composition comprising: 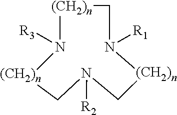
wherein R₁, R₂ and R₃ are independently selected from the group consisting of a proton, an alkyl, an ether, an alcohol, a carboxylic acid, an aryl, an amino acid, or a peptide;
wherein each n varies independently and is an integer equal to either 1 or 2; and
wherein the structure includes an interchelated metal ion selected from the group consisting of iron, ruthenium, osmium, cobalt, rhodium, iridium, nickel, palladium, platinum, copper, chromium, gallium, molybdenum, manganese, and tungsten.

13. A chemical composition as in claim 12 wherein the structure further comprises an interchelated Rh(III) metal bound to the one or more nitrogen atoms.

14. A chemical composition as in claim 12 wherein the structure further comprises an interchelated Ru(III) metal bound to the one or more nitrogen atoms.

15. A chemical composition comprising: 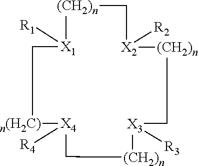
wherein R₁, R₂, R₃ and R₄ are independently selected from the group consisting of a proton, an alkyl, an ether, an alcohol, a carboxylic acid, an aryl, an amino acid, a peptide, or null;
wherein X₁, X₂, X₃, and X₄ are independently either sulfur or nitrogen;
wherein if X₁ is sulfur, R₁ is null;
wherein if X₂ is sulfur, R₂ is null;
wherein if X₃ is sulfur, R₃ is null;
wherein if X₄ is sulfur, R₄ is null; and
wherein the structure includes an interchelated metal ion selected from the group consisting of iron, ruthenium, osmium, cobalt, rhodium, iridium, nickel, palladium, platinum, copper, chromium, gallium, molybdenum, manganese, and tungsten.

16. A chemical composition as in claim 15 wherein the structure further comprises an interchelated Rh(III) metal bound to the one or more nitrogen atoms.

17. A chemical composition comprising: 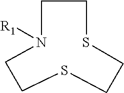
wherein the structure includes an interchelated Rh(III) metal bound to the nitrogen atom and one or both of the sulfur atoms.

18. A chemical composition comprising:

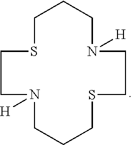

19. The composition of claim 18 further comprising: 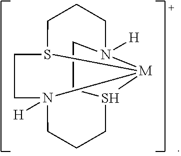
wherein M is a transition metal.

20. The composition of claim 19 wherein M comprises rhodium (III) according to the structure: 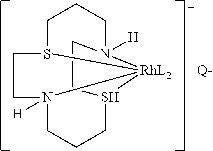
wherein L is a counter ion selected from the group consisting of chloride, bromide, iodide, and alkoxide; and
wherein Q- is a counterion selected from the group consisting of chloride, bromide, iodide, and alkoxide.

21. A composition comprising: 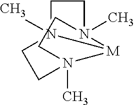
wherein M comprises a metal ion selected from the group consisting of iron, ruthenium, osmium, cobalt, rhodium, iridium, nickel, palladium, platinum, copper, chromium, gallium, molybdenum, manganese, and tungsten.

22. The composition of claim 21, wherein M comprises rhodium (III).
